(19) United States
(12) Patent Application Publication
(10) Pub. No.: US 2011/0218409 A1
(43) Pub. Date: Sep. 8, 2011

(54) Device and method for measuring sleep apneas

(76) Inventors: Andreas Kugler, Alfdorf (DE); Ulrich Ladstaetter, Weinstadt (DE); Patrick Stihler, Ostfildern (DE)

(21) Appl. No.: 12/932,586

(22) Filed: Feb. 28, 2011

(30) Foreign Application Priority Data
Mar. 4, 2010 (DE) 10 2010 002 562.3

(51) Int. Cl. A61B 5/00

(52) U.S. Cl. 600/301

(57) ABSTRACT

A device is implemented as a functional patch, for measuring sleep apneas, which device includes: an acoustic sensor for measuring respiration and snoring noises; an optical sensor for measuring a saturation of oxygen in the blood of a human; and an adhesive patch which carries the acoustic sensor and the optical sensor. Analysis of the measuring signals and display of the sleep apneas are alternately performed in the functional patch or in an external unit, to which the data are transmitted wirelessly.

### BACKGROUND OF THE INVENTION

1. Field of the Invention

The present invention relates to a device and a method for measuring sleep apneas.

2. Description of the Related Art

One possibility for recognizing sleep apneas uses the determination of the blood oxygen content in connection with the observation of respiration and snoring noises. The blood oxygen content of human blood may be ascertained via the pulse oximetry method. The oxygen content is concluded by measuring the light absorption or the light remission during fluoroscopy of the skin (percutaneous). The measurement is performed using a saturation transducer, a clip, or an adhesive sensor, on an easily accessible body part, preferably on a finger, a toe, or at the earlobe. The measurement may also be performed on the neck, the temple, or the forehead via a patch. The sensor has two light sources which glow in a defined red and infrared range on one side and a photosensor on the other side. A differing absorption, which the photo sensor measures, arises for the transmitted red light through the differing coloration of the hemoglobin containing oxygen compared to hemoglobin which does not contain oxygen. Three values are measured, the absorption of the light in the 660 nm range and in the 940 nm range as a differential measurement, and for calibration without the radiation of the measuring light sources, using only ambient light. The measurement detects the pulsating through-flowing blood and not the tissue and the vessels. On the basis of a comparison of the measurement result to a reference table, a monitoring monitor ascertains the percentage component of the red blood cells which is saturated. The partial oxygen saturation is thus ascertained. Typical values are between 96% and 100% in a healthy person. The respiration and snoring are observed using a microphone. Thus, published German patent application document DE 41 38 702 A1 describes a method and a device for diagnosing apnea and simultaneously establishing other illnesses. The microphone, the optical sensors, and further sensors are all connected via cables to a detection and storage device, which continuously stores the measured data. The collected data are transmitted to an external PC for analysis. Such systems are being used in clinics and doctor's offices. These systems are awkward and inflexible in wearing comfort and also restrict the quality of life for the user. In addition, this type of indication is immediately connected to high costs. Furthermore, functional patches having a microphone and functional patches having an optical sensor are known, both to be connected via a data cable. Such patches are used individually for sleep observation.

### BRIEF SUMMARY OF THE INVENTION

In contrast, the device and the method for measuring sleep apneas according to the present invention have the advantage that the sensors are integrated in wireless functional patches, which means a significant improvement in comfort compared to sensors having cables. A further advantage is that both the acoustic sensor and also the optical sensor are contained in a single patch. This increases the diagnostic reliability compared to the functional patches based on the isolated individual measuring methods. An advantage of the present invention is the comfortable measurement of the oxygen saturation and the sleep noises or the sleep apnea potential without complex, bulky equipment, and without restriction of the mobility. Prescreening is possible without time-consuming and costly visits to the physician or at least with a smaller number thereof. A measurement may be performed at any desired time without bulky equipment being cumbersome during sleep, in the case of apnea in particular. The patch is preferably offered as a disposable patch or as a multiuse patch.

### BRIEF DESCRIPTION OF THE DRAWINGS

FIG. 1 shows a schematic view of preferred locations of the application of a device according to the present invention on the human body.

FIG. 2 shows a schematic view of a device according to one specific embodiment of a first variant of the present invention.

FIG. 3 shows a schematic view of a device according to one specific embodiment of a second variant of the present invention.

FIG. 4 shows a schematic view of a device according to one specific embodiment of a third variant of the present invention.

FIG. 5 shows a side view of the device from FIG. 2.

FIG. 6 shows a flow chart of the method according to one specific embodiment of a first variant of the present invention.

FIG. 7 shows a flow chart of the method according to one specific embodiment of a second variant of the present invention.

FIG. 8 shows a flow chart of the method according to one specific embodiment of a third variant of the present invention.

### DETAILED DESCRIPTION OF THE INVENTION

FIG. 1 shows a device 10 according to the present invention having preferred locations 11 of the application on human body 12. The application includes sticking on a patch, which is packaged in the form of a commercially-available patch and may be stuck onto the body of the patient and detects the sleep/respiration noises and simultaneously also the blood oxygen saturation.

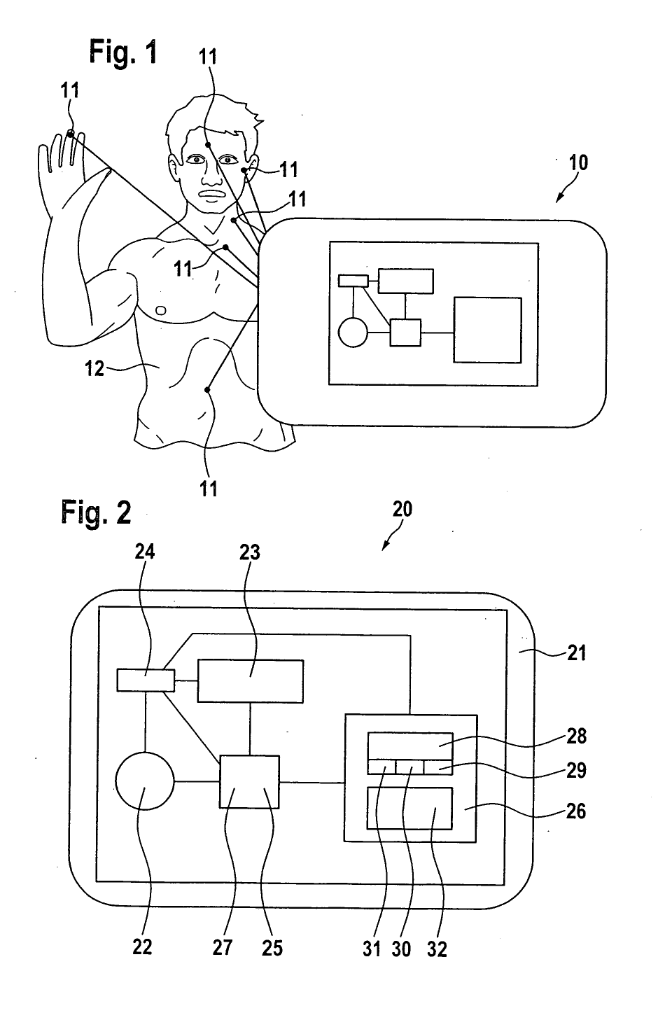

FIG. 2 shows a device 20 for measuring sleep apneas according to one specific embodiment of a first variant of the present invention. An adhesive patch 21 carries an electret microphone 22, an optical sensor 23, a battery 24, a detection unit 25, and an information'transmission unit 26. Battery 24 is a microbattery, for example, a thin-film battery, or a paper battery, and is connected to electret microphone 22, optical sensor 23, detection unit 25, and information transmission unit 26 to supply them with electrical voltage. Optical sensor 23 contains an LED in integrated form, which may alternately emit one of two wavelengths at 660 nm and 940 nm, and a detector, in the form of a photodiode here. An acoustic sensor is electret microphone 23 here, but other microphones such as MEMS microphones are also possible. Detection unit 25 is used for controlling the LED and detecting the signals of electret microphone 22 and optical sensor 23.

The first variant of the present invention is an integrated approach, the signals detected by sensors 22 and 23 are collected and analyzed within device 20 and the analysis results are also displayed using device 20. For this purpose, detection unit 25 has a data memory and is designed as an analysis unit 27. Information transmission unit 26 has an LED display 28 having three color fields 29, 30, 31 and a display screen 32. The LED may be implemented as an OLED. The colors of the color fields are preferably red, yellow, and green. Display screen 32 is an LCD display, however, an electrophoretic display (EPD) is also possible. Information transmission unit 26 transmits analyzed information about sleep apneas to a patient using display screen 32.

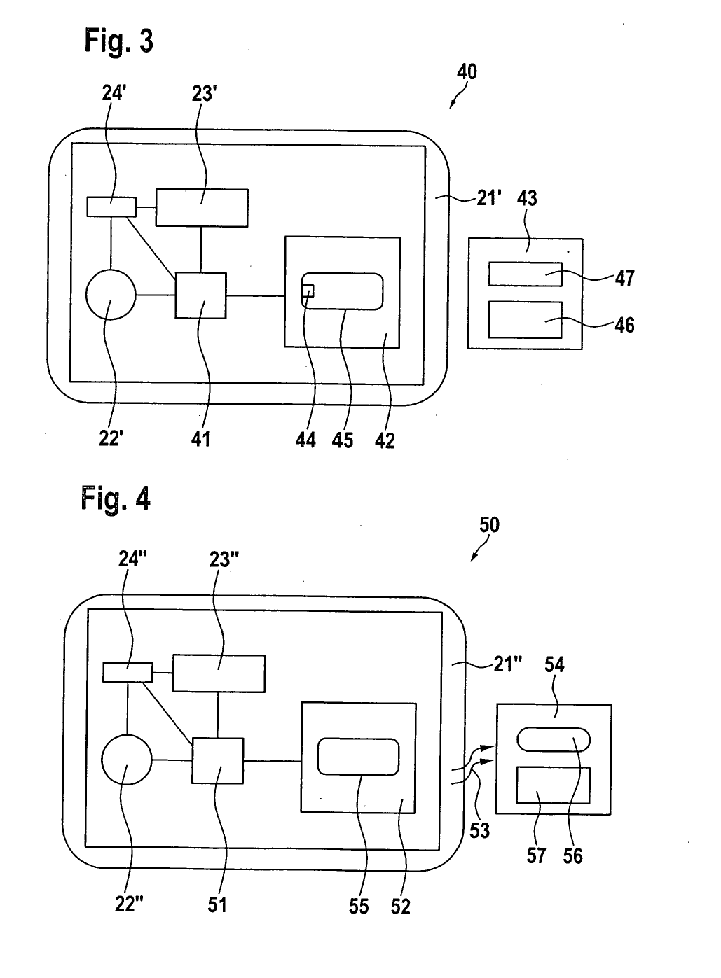

FIG. 3 shows a device 40 for measuring sleep apneas according to one specific embodiment of a second variant of the present invention. The elements known from FIG. 1 have identical reference numerals, which are provided with apostrophes, however. An adhesive patch 21′ carries an electret microphone 22′, an optical sensor 23′, a battery 24′, a detection unit 41, and an information transmission unit 42. Battery 24′ is connected to electret microphone 22′, optical sensor 23′, detection unit 41, and information transmission unit 42 to supply them with electrical voltage. Optical sensor 23′ contains an LED in integrated form, which may alternately emit one of two wavelengths at 660 nm and 940 nm, and a detector in the form of a photodiode. Detection unit 41 is used for controlling the LED and detecting the signals of electret microphone 22′ and optical sensor 23′.

The second variant of the present invention is an approach in which the signals detected by sensors 22′ and 23′ are collected within device 40, but the signals are analyzed and the analysis results are displayed using an external analysis and display device 43. For this purpose, detection unit 41 has a data memory. Information transmission unit 42 has an RFID chip 44 and a printed RFID antenna 45. Information transmission unit 42 transmits the measured data, which are collected in detection unit 41, after a measuring period to external analysis and display device 43, in which the corresponding data are read out in an RFID readout station 47 of external analysis and display device 43 using the RFID method. External analysis and display device 43 has a display screen 46 to display the analyzed information about sleep apneas to a physician or a patient.

One alternative exists in the event of sufficiently great computing power of detection unit 41, which then operates as a detection and analysis unit as in FIG. 1. The analyzed information is correspondingly transmitted to an external RFID readout station with the aid of RFID for display in an external display unit.

A device 50 for measuring sleep apneas according to one specific embodiment of a third variant of the present invention is shown in FIG. 4. Elements known from FIG. 1 have identical reference numerals, which are provided with a quotation mark, however. An adhesive patch 21″ carries an electret microphone 22″, an optical sensor 23″, a battery 24″, a detection unit 51, and an information transmission unit 52. Battery 24″ is connected to electret microphone 22″, optical sensor 23″, detection unit 51, and information transmission unit 52, in order to supply them with electrical voltage. Optical sensor 23″ contains an LED in integrated form, which may alternately emit one of two wavelengths at 660 nm and 940 nm, and a detector in the form of a photodiode. Detection unit 51 is used for controlling the LED and detecting the signals of electret microphone 22″ and optical sensor 23″.

The third variant of the present invention is an approach in which the signals detected by sensors 22″ and 23″ during the measurement are transmitted via radio signals 53 to an external analysis and display device 54, in which the signals are analyzed and the analysis results are displayed. For this purpose, information transmission unit 52 has a transmitting antenna 55. External analysis and display device 54 has a receiving antenna 56 to receive the measured data and a display screen 57 to display the analyzed information about sleep apneas to a physician or a patient.

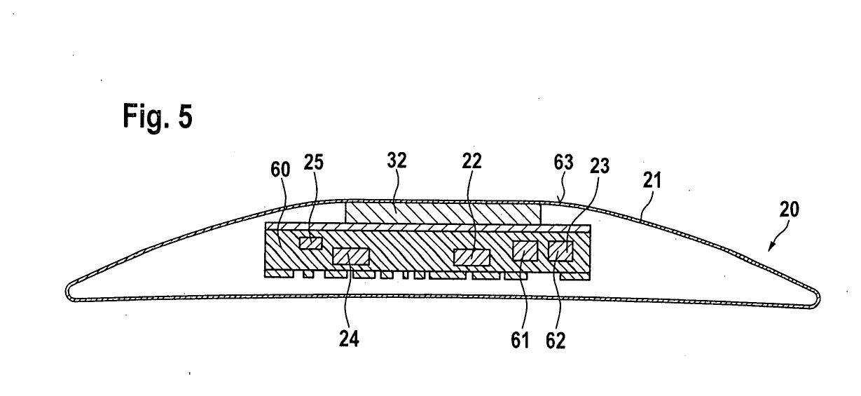

FIG. 5 shows a side view of the device from FIG. 2. Electret microphone 22, optical sensor 23, battery 24, and detection unit 25 are integrated in a film 60 inside adhesive patch 21. Optical sensor 23 contains LED 61 and photodiode 62. Display 32 is situated on top side 63 of adhesive patch 21.

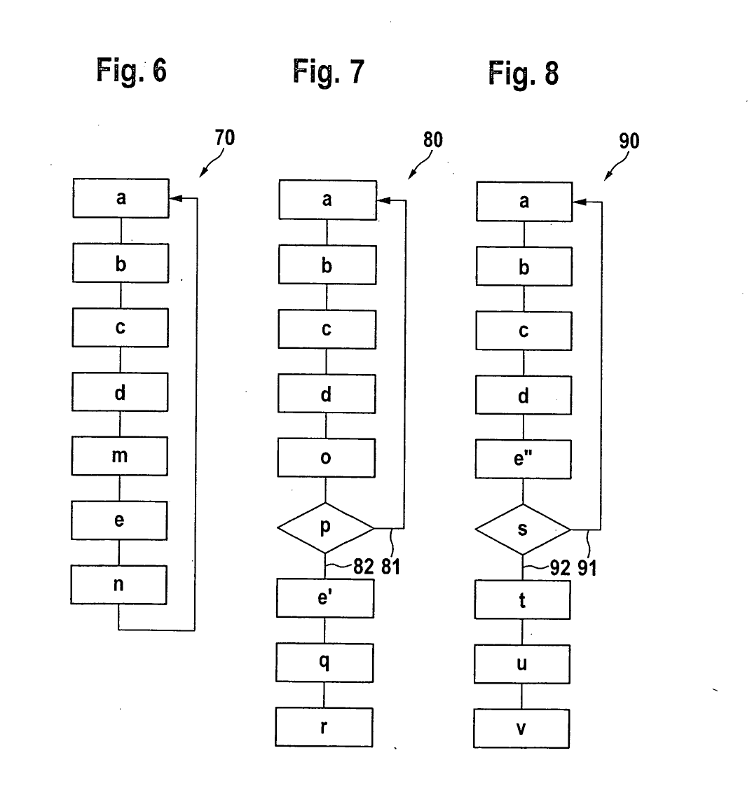

The mode of operation of the various variants of the device will be described on the basis of FIGS. 6 through 8, which show corresponding variants of the method in the form of flow charts. Reference is made to FIGS. 2 through 5.

FIG. 6 shows a flow chart 70 of the method according to one specific embodiment of a first variant of the present invention. The method begins with method step a) performing an optical calibration measurement using optical sensor 23 with turned-off LED 61 of optical sensor 23. The sequence continues with method step b) first optical measurement of first measured values using optical sensor 23 with LED 61 operating at the first wavelength in the range around 660 nm to ascertain a blood oxygen concentration; and method step c) second measurement of second measured values using optical sensor 23 with LED 61 operating at a wavelength in the range around 940 nm to ascertain a blood oxygen concentration. The sequence continues with step d) performing an acoustic measurement of third measured values using electret microphone 22 to ascertain a respiration. Method steps a) through d) may be performed in arbitrary sequence and are identical in all variants of the method. In method step m), the measured values are stored in the data memory of detection unit 25 and the measured values are analyzed and values of the blood oxygen concentration and values of the respiration are ascertained in analysis unit 27. Subsequently, the sequence continues with method step e) transmitting the values of the blood oxygen concentration and values of the respiration to information transmission unit 26. The sequence continues with method step n) displaying the values of the blood oxygen concentration and the values of the respiration with the aid of LED display 28 and in display screen 32. The method starts over with method step a).

The data or results ascertained by analysis unit 27 may be displayed by optical imaging methods on device 20 with the aid of LED display 28 and in display screen 32. Color coding in the form of a traffic signal display is performed using three color fields 29, 30, 31 of LED display 28, in the example, green, yellow, and red corresponding to an ascertained health status, the presence of heart sounds also being able to be considered. An alternative to the LED display are electrochemical color reactions by application of an electrical voltage. As in the case of the LED display, a color change indicates the occurrence or non-occurrence of an event. Characters and numbers are displayed directly in display screen 32, e.g., number of apneas in the running measurement.

FIG. 7 shows a flow chart 80 of the method according to one specific embodiment of a second variant of the present invention. The method begins with method step a) performing an optical calibration measurement using optical sensor 23′ with turned-off LED of optical sensor 23′. The sequence continues with method step b) first optical measurement of first measured values using optical sensor 23′ at the first wavelength to ascertain a blood oxygen concentration; and method step c) second measurement of second measured values using optical sensor 23′ at a second wavelength to ascertain a blood oxygen concentration. The sequence continues with method step d) performing an acoustic measurement of third measured values using electret microphone 22′ to ascertain a respiration. Method steps a) through d) may be performed in arbitrary sequence. The sequence continues in method step o) storing the measured values in the data memory of detection unit 25. The sequence subsequently continues in method step p) checking whether the measurement has ended according to a predetermined measuring duration. If not, the sequence branches into branch 81 after a) and the measurement is continued. If the predetermined measuring duration is reached, the sequence branches into branch 82 and continues with method step e′) transmitting the measured values from detection unit 41 to information transmission unit 26 and relaying them using an RFID method to external analysis and display device 43. The sequence continues with method step q) analyzing the measured values and ascertaining values of the blood oxygen concentration and values of the respiration in external analysis and display device 43. The sequence finally performs method step r) displaying the values of the blood oxygen concentration and the values of the respiration with the aid of analysis and display device 43.

The data are preferably read out via RFID at home, in a pharmacy, or in a doctor's office. The data may be transmitted after the readout by telephone, via Bluetooth, or via WLAN.

A flow chart 90 of the method according to one specific embodiment of a second variant of the present invention is shown in FIG. 8. The method begins with method step a) performing an optical calibration measurement using optical sensor 23″ with turned-off LED of optical sensor 23″. The sequence continues with method step b) first optical measurement of first measured values using optical sensor 23″ at the first wavelength to ascertain a blood oxygen concentration; and method step c) second measurement of second measured values using optical sensor 23″ at a second wavelength to ascertain a blood oxygen concentration. The sequence continues with method step d) performing an acoustic measurement of third measured values using electret microphone 22″ to ascertain a respiration. Method steps a) through d) may be performed in arbitrary sequence. The sequence continues in method step e″) transmitting the measured values from detection unit 41 to information transmission unit 26 and relaying them to external analysis and display device 54 via radio, for example, Bluetooth. The measured values are buffered therein. The sequence continues with method step s) checking whether the measurement has ended according to a predetermined measuring duration. If not, the sequence branches into branch 91 after a) and the measurement is continued. If the predetermined measuring duration is reached, the sequence branches into branch 92 and continues with method step t) storing the measured values in the data memory of external analysis and display device 54. Subsequently, the sequence continues with method step u) analyzing the measured values and ascertaining values of the blood oxygen concentration and values of the respiration in external analysis and display device 54. Finally, the sequence continues with method step v) displaying the values of the blood oxygen concentration and the values of the respiration with the aid of analysis and display device 54.

The device according to the present invention is manufactured as a functional patch, which is packaged in the form of a commercially-available patch and may be stuck on the body of the patient. Using this patch, the sleep/respiration noises and simultaneously also the blood oxygen saturation are detected. The measured data are alternately transmitted wirelessly online or stored in the patch or by a receiving station according to the variant. In the case of an online measurement and transmission, the measured values are continuously retrieved or transmitted at defined time intervals and recorded, i.e., stored, analyzed, and displayed by external analysis and display device 54, which is spatially separated from the functional patch. In the case of storage, the data are also detected at defined time intervals, e.g., every two seconds, but are only stored after the defined measuring time period, e.g., overnight, via passive/active radio retrieval (RFID or active transmission) and relayed to a receiving station or alternately analyzed by the analysis chip and subsequently relayed to the receiving station. Alternatively, in the case of the storage method, after analysis by the analysis chip, the result may be displayed via visual display elements, e.g., electrophoretic, LED, OLED, LCD.

What is claimed is:

1. A device for measuring sleep apneas comprising:
an acoustic sensor for measuring respiration and snoring noises;
an optical sensor for measuring a saturation of oxygen in the blood of a human; and
an adhesive patch which carries the acoustic sensor and the optical sensor.

2. The device as recited in claim 1, further comprising:
a voltage supply unit.

3. The device as recited in claim 1, further comprising:
a detection unit.

4. The device as recited in claim 3, further comprising:
an information transmission unit.

5. The device as recited in claim 4, wherein the acoustic sensor and the optical sensor are embedded in a film.

6. The device as recited in claim 5, further comprising:
a storage unit for storing measured values.

7. The device as recited in claim 6, wherein the information transmission unit has a display unit.

8. The device as recited in claim 7, wherein the display unit has a display screen.

9. The device as recited in claim 7, wherein the display unit has a traffic signal display having three colors.

10. The device as recited in claim 5, wherein the information transmission unit has a transmitting unit for transmitting measured values to an external receiving unit.

11. The device as recited in claim 9, wherein the information transmission unit has an RFID chip and an RFID antenna.

12. A method for measuring sleep apneas using an acoustic sensor and an optical sensor, comprising:
a) performing an optical calibration measurement;
b) performing first optical measurement of first measured values at a first wavelength to ascertain a blood oxygen concentration;
c) performing second measurement of second measured values at a second wavelength to ascertain a blood oxygen concentration;
d) performing an acoustic measurement of third measured values to ascertain a respiration; and
e) transmitting the measured values or values of the blood oxygen concentration and values of the respiration;
wherein the method steps a) through d) are performed in arbitrary sequence.

13. The method as recited in claim 12, wherein the values of the blood oxygen concentration and the values of the respiration are stored.

14. The method as recited in claim 12, wherein the values of the blood oxygen concentration and the respiration are transmitted to a display unit.

15. The method as recited in claim 12, wherein the values of the blood oxygen concentration are ascertained from the first and second measured values and the values of the respiration are ascertained from the third measured values.
